(19) United States
(12) Patent Application Publication
(10) Pub. No.: US 2011/0056854 A1
(43) Pub. Date: Mar. 10, 2011

(54) Method For Preparing A Lubricating Film, Solid Support Thus Obtained And Preparation Kit

(75) Inventors: Thomas Berthelot, Villebon Sur Yvette (FR); Fabien Nekelson, Paris (FR); Sébastien Roussel, Soisy Sur Seine (FR); Matthieu Laurent, Vaucresson (FR)

(73) Assignee: Commisssariat A L'Energie Atomique Et Aux Energies Alternatives, Paris (FR)

(21) Appl. No.: 12/822,102

(22) Filed: Jun. 23, 2010

(30) Foreign Application Priority Data
Jun. 23, 2009 (FR) 09 54281

(51) Int. Cl. B65D 85/00; B05D 3/00; F16C 33/20; G01N 33/48; F16N 15/00

(52) U.S. Cl. 206/223; 427/299; 508/100; 422/68.1; 184/14

(57) ABSTRACT

The present invention concerns a method for the lubrication of the surface of a solid by grafting a polymer gel, and for impregnation with a lubricating composition, and a kit to prepare a solid comprising a surface lubricated with a polymer gel.

### TECHNICAL FIELD

The present invention concerns the field of the lubrication of solid substrate surfaces, notably to resist friction. The invention particularly concerns lubrication means applicable by coating, spraying or dipping the surface. It more particularly concerns the use of solutions appropriately selected to allow the simple and reproducible formation of lubricating (or lubricant) films by surface coating, spraying or dipping.

### PRIOR ART

At the current time, there exist several techniques allowing the lubrication of parts in contact with each other, these techniques being briefly recalled below.

The method to form a coating known as “spin coating” does not require any particular affinity between the deposited lubricating molecules and the substrate of interest. This is also the case for related techniques to form coatings by dip coating or by spray coating. The cohesion of the deposited film is essentially based on interactions between its constituents. The film, for example, may be crosslinked after deposit to improve its stability. These techniques are most versatile, applicable to any type of surface to be coated, and very reproducible. However, they do not allow any effective grafting between the film and the substrate (since it involves mere physical adsorption) and the thicknesses produced are ill-controlled, notably the thinnest deposits (of less than 20 nanometres). In addition, spin coating techniques only allow uniform depositing when the surface to be coated is essentially planar.

Depositing in solution is also possible but it is not satisfactory either, owing to the low resistance of the deposit.

Plasma depositing is based on the principle of generating unstable forms of a precursor close to the surface to be coated, these forms progressing towards the formation of a film on the substrate. This type of method was notably used to create a lubricating layer on the surface of a polymer material [Satyanarayana, N. et al. Applied Physics Letters 2008, 93, 261906]. Not all substrates, notably the most sensitive, lend themselves to this type of treatment.

The adsorption of a compound, either allowing modification of the surface charge or the obtaining of a neutral hydrophobic surface, has also been proposed as base structure for lubrication. Polymer brushes are then deposited in solution and are chosen in view of the electrostatic or hydrophobic nature of the compound adsorbed on the surface. For example, mica has been thus modified by depositing polylysine before the depositing of the lubrication layer (i.e. lubricin) [Zappone, B. et al. Biophysical Journal 2007, 92, 1693-1708]. Here again the deposit of lubricating film is lax.

Some proposed methods do not allow the ensuring of durable resistance of the lubricating film. Up until now, there has effectively often been the risk that this film diffuses into its environment. In addition, some methods are not applicable to all types of substrates since they are too aggressive.

The lubricating compounds frequently used in pharmaceutical, food and cosmetic industries, irrespective of their mode of application, correspond to silicon oils such as dimethicone (polydimethylsiloxane (PDMS)) or cyclodimethicone (octamethylcyclotetrasiloxane) or different types of grease.

For these industries, it is important that the lubricants used meet quality and tolerance criteria laid down by legislation. Siloxanes have relative innocuousness and it is therefore desirable to be able to provide these industries with systems having tribological properties of interest and whose lubricating means are not toxic and do not significantly release themselves into the environment of the lubricated material.

It is also desirable that the decomposition products of the lubrication means do not appear in significant amount in the environment of the material when in use, and are not toxic. It is recommended that the lubrication means should be adapted to an aqueous environment.

### DESCRIPTION OF THE INVENTION

With the present invention, it is possible to solve the disadvantages of the methods and coatings of the prior art. It notably sets itself apart from the prior art in that it allows a substantial improvement in the tribological properties of materials used in the medical, food, cosmetic fields in particular and in precision mechanics, whilst guaranteeing the resistance of the lubrication means. In addition, it can be adapted to different lubricating compositions. The proposed method therefore allows the grafting of lubricating films onto surfaces of varied types.

The invention concerns a method for lubricating a surface of a solid by covalent grafting of a colloidal material, and more particularly of a polymer gel.

A colloidal material comprises two separate phases whose particles in one discontinuous phase are much smaller, and are dispersed in the other phase. A gel corresponds to a colloid which has a solid phase within which there exists a liquid dispersed phase. Therefore, according to the International Union of Pure and Applied Chemistry (IUPAC), a polymer gel is defined as a gel in which the continuous phase is formed of polymer network.

The invention therefore concerns a method for lubricating a surface of a solid, comprising the steps consisting of:

a) grafting on said surface a polymer organic film whose first unit derives from an adhesion primer, and of which at least one other unit derives from a hydrophilic monomer that is radically polymerizable, and optionally

b) impregnating the polymer organic film of step (a) with a lubricating composition.

By “adhesion primer”, under the present invention is meant any organic molecule which, under certain non-electrochemical or electrochemical conditions, is able to form either radicals or ions, cations in particular, and is therefore able to take part in chemical reactions. Said chemical reactions may in particular concern chemisorption, notably chemical grafting or electrografting. Therefore, said adhesion primer, under non-electrochemical or electrochemical conditions, is capable of chemisorbing on the surface, notably by radical reaction, and of having another reactive function with respect to another radical after this chemisorption. The adhesion primer is a cleavable aryl salt.

The invention particularly concerns a method for lubricating a surface of a solid, comprising the steps consisting of:

a) grafting on said surface a polymer organic film consisting of polymers grafted on said surface, each polymer having a first unit directly bonded to said surface, derived from a cleavable aryl salt, and at least one other unit of the polymer chain derived from a radically polymerizable hydrophilic monomer, and optionally

b) impregnating the polymer organic film of step (a) with a lubricating composition.

As a variant, the lubrication method according to the present invention has a non-optional impregnation step with a lubricating composition. Therefore the present invention also concerns a method for lubricating a surface of a solid, comprising the steps consisting of:

a) grafting on said surface a polymer organic film consisting of polymers grafted on said surface, each polymer having a first unit directly bonded to said surface, derived from a cleavable aryl salt, and at least one other unit of the polymer chain derived from a radically polymerizable hydrophilic monomer, and

b) impregnating the polymer organic film of step (a) with a lubricating composition.

Advantageously, the cleavable aryl salt is chosen from the group consisting of aryl diazonium salts, aryl ammonium salts, aryl phosphonium salts, aryl iodonium aryl salts and aryl sulfonium salts. In these salts, the aryl group is an aryl group which can be represented by R such as defined below.

Among the cleavable aryl salts, particular mention may be made of the compounds of following formula (I):

R—N₂⁺,A⁻ (I)

wherein:
A is a monovalent anion, and
R is an aryl group.

As aryl group of cleavable aryl salts and notably of the above formula (I) compounds, advantageous mention may be made of aromatic or heteroaromatic carbon structures, optionally mono- or polysubstituted, consisting of one or more aromatic or heteroaromatic cycles each comprising 3 to 8 atoms, the heteroatom(s) possibly being N, O, P or S. The substituent(s) may contain one or more heteroatoms, such as N, O, F, Cl, P, Si, Br or S, and C₁ to C₆ alkyl groups or C₄ to C₁₂ thioalkyl groups in particular.

Among the cleavable aryl salts and notably the above formula (I) compounds, R is preferably chosen from among the aryl groups substituted by electron-attracting groups such as NO₂, COH, ketones, CN, CO₂H, NH₂ (in NH₃⁺ form), esters and halogens. The aryl-type R groups that are particularly preferred are benzene and nitrobenzene radicals, optionally substituted.

Among the above formula (I) compounds, A may notably be chosen from among inorganic anions such as halides e.g. I⁻, Br⁻ and Cl⁻, halogenoborates such as tetrafluoroborate, perchlorates and sulfonates, and from among organic anions such as alcoholates and carboxylates.

As formula (I) compounds, it is of particular advantage to use a compound chosen from the group consisting of 4-nitrobenzenediazonium tetrafluoroborate, tridecylfluorooctylsulfamylbenzene diazonium tetrafluoroborate, phenyldiazonium tetrafluoroborate, 4-nitrophenyldiazonium tetrafluoroborate, 4-bromophenyldiazonium tetrafluoroborate, 4-aminophenyldiazonium chloride, 2-methyl-4-chlorophenyldiazonium chloride, 4-benzoylbenzenediazonium tetrafluoroborate, 4-cyanophenyldiazonium tetrafluoroborate, 4-carboxyphenyldiazonium tetrafluoroborate, 4-acetamidophenyldiazonium tetrafluoroborate, 4-phenylacetic diazonium acid tetrafluoroborate, 2-methyl-4-[(2-methylphenyl)diazenyl]benzenediazonium sulfate, 9,10-dioxo-9,10-dihydro-1-anthracenediazonium chloride, 4-nitronaphtalenediazonium tetrafluoroborate and naphtalenediazonium tetrafluoroborate.

By “radically polymerizable hydrophilic monomer” is meant a hydrophilic compound comprising an ethylene bond, different from the adhesion primer. Said compounds advantageously comprise at least one polar function such as an alcohol, an ether, an ester or a carboxylic acid. In the meaning of the invention, a hydrophilic compound typically corresponds to a water-soluble compound in any proportion, and more particularly whose solubility is greater than 0.1 M under normal temperature and pressure conditions (NTP). According to the invention, and unless indicated otherwise, NTP conditions relate to a temperature of 25° C. and a pressure of 1.10⁵ Pa.

Under the invention, it is possible to use hydrophilic monomers meeting the following formula (II):

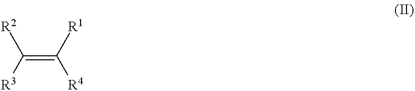

wherein the groups R₂ to R₄, the same or different, represent a non-metallic monovalent atom such as a halogen atom, a hydrogen atom, a saturated or unsaturated chemical group such as an alkyl or aryl group, a hydrophilic polymer chain, a nitrile, a carbonyl, an amine, an amide, a —COOR₅ group in which R₅ is a hydrogen atom or a C₁-C₁₂ alkyl group and preferably C₁-C₆,

wherein the R₁ group represents a carboxylic acid, a hydrophilic polymer chain or a —COOR₅ group in which R₅ is a hydrophilic polymer chain.

Advantageously the groups R₂ to R₄ preferably comprise one or more oxygen or nitrogen atoms in ionizable form in an aqueous medium, and notably alcohol, ether, ester, carboxylic acid or amine functions.

Among the hydrophilic monomers of formula (II), a distinction can be made between the “vinyl terminal hydrophilic macromolecules” which correspond to molecules with high molecular weight whose structure is essentially formed of multiple repeat units, derived actually or conceptually from molecules of relative low molecular weight. This is notably the case with compounds in which R₁ comprises a hydrophilic polymer chain, such as a polyether, in which the hydrophilic unit is typically repeated between 10 and 100 times and, in particular, between 10 and 40 times. The hydrophilic units typically correspond to carbon units comprising one or more oxygen or nitrogen atoms optionally in the form of ions.

As hydrophilic monomers which can be used under the present invention, particular mention may be made of acrylic acid, hydroxyethyl methacrylate (HEMA), and vinyl terminal hydrophilic macromolecules such as polyethylene glycol methacrylate (PEGm).

Advantageously, the hydrophilic monomers have lubricating properties. Therefore typically for one same surface, when deposited or grafted, they will ensure lubrication that is at least equal to that of a conventional silicon oil and notably of DM 1000 type. In this case, the impregnation of the grafted polymer organic film of which some units are derived from said hydrophilic monomers, is optional. In other words, in this case step (b) of the method according to the invention is optional.

The invention also applies to a mixture of two, three, four or more monomers chosen from among the previously described monomers. In this case, the film is said to be “heterogeneous” since it contains units derived from monomers of different type. A film solely containing units derived from a single type of monomer is said to be “homogeneous”.

Typically, in the inventive method, at least one monomer is a vinyl terminal hydrophilic macromolecule. If one of the monomers corresponds to a vinyl terminal macromolecule, the proportion thereof is preferably equal to or less than that of other monomers.

According to one particular embodiment, the film contains units derived from a first monomer of vinyl terminal hydrophilic macromolecule type, and units derived from a second monomer which is not a vinyl terminal macromolecule.

More particularly, it is possible to produce heterogeneous films containing units derived from a first radically polymerizable hydrophilic monomer and units derived from a second monomer of radically polymerizable hydrophobic type. The radically polymerizable hydrophobic monomers correspond to hydrophobic compounds comprising an ethylene bond different from the adhesion primer. Said compounds comprise apolar functions such as an alkyl group, an aromatic residue, a fluoroalkyl group, a polymer chain such as a polyalkylsiloxane or a polypropyleneglycol (PPG). It may for example be a propyleneglycol methacrylate (PPGm). In the meaning of the invention, a hydrophobic compound typically corresponds to a compound considered to be water-insoluble, more particularly whose solubility is less than 0.1 M under normal temperature and pressure conditions (NTP).

Therefore the organic film used under the present invention is essentially polymer or copolymer, derived from several monomer units of identical or different chemical species, and from adhesion primer molecules. The films obtained using the method of the present invention are “essentially” of polymer type insofar as the film also incorporates species derived from the adhesion primer and not only from the monomers present. The organic film in the present invention has a sequence of monomer units in which the first unit consists of a derivative of the adhesion primer or results from an adhesion primer, the other units indifferently being derivatives or resulting from the adhesion primers and/or from the monomers such as previously defined. The units of the organic film from the latter therefore result from polymerization, notably radical polymerization, of the elements present and chosen from among the adhesion primers and the radically polymerizable monomers different from the adhesion primer.

Typically, the grafting used at step (a) of the method of the invention is chemical grafting.

The term “chemical grafting” notably refers to the use of extremely reactive (typically radical) molecular entities capable of forming bonds of covalent bond type with a surface of interest (i.e. surface of the solid), said molecular entities being generated independently of the surface on which they are intended to be grafted. Therefore the grafting reaction leads to the formation of covalent bonds between the surface to be coated with an organic film and the derivative of the adhesion primer.

By “adhesion primer derivative” under the present invention is meant a chemical unit resulting from the adhesion primer, after the latter has reacted with the surface by chemical grafting, and optionally with another chemical compound via radical reaction, said other chemical compound giving the second unit of the organic film. Therefore the first unit of the organic film is a derivative of the adhesion primer which reacted with the surface and with another chemical compound.

Advantageously, grafting step (a) comprises the steps consisting of:

a₁) contacting said surface with a solution S₁ comprising at least one adhesion primer and at least one radically polymerizable hydrophilic monomer;

b₁) subjecting said solution S₁ to non-electrochemical conditions enabling the formation of radical entities from said adhesion primer.

Any surface, whether inorganic or organic, having one or more atoms or groups of atoms possibly being involved in a radical addition or substitution reaction, such as CH, carbonyls (ketone, ester, acid, aldehyde), —OH, ethers, amines, halogens, such as F, Cl, Br, is notably concerned by the present invention.

Surfaces of inorganic type can notably be chosen from among conductive materials such as metals, noble metals, oxidized metals, transition metals, metal alloys and for example Ni, Zn, Au, Pt, Ti or steel. They may also be semiconductor materials such as Si, SiC, AsGa, Ga, etc. It is also possible to apply the method to non-conductive surfaces such as non-conductive oxides e.g. SiO₂, Al₂O₃ and MgO. More generally, an inorganic surface can for example be formed of an amorphous material such as glass generally containing silicates, or a ceramic, crystalline such as diamond, graphite possibly being organized to a greater or lesser extent such as graphene, highly oriented pyrolytic graphite (HOPG), or carbon nanotubes.

As surface of organic type, particular mention may be made of natural polymers such as latex or rubber, or artificial polymers such as derivatives of polyamide or polyethylene, and notably polymers having bonds of π type such as polymers carrying ethylene bonds, carbonyl groups, imine groups. It is also possible to apply the method to more complex organic surfaces such as leather surfaces containing polysaccharides, such as cellulose for wood or paper, artificial or natural fibres such as cotton or felt, and fluorinated polymers such as polytetrafluoroethylene (PTFE), or to polymers carrying basic groups such as tertiary or secondary amines for example pyridines e.g. poly-4 and poly-2-vinylpyridines (P4VP and P2VP) or more generally polymers carrying aromatic and nitrogen aromatic groups.

More particularly, the method applies to biocompatible polymer surfaces, thermopolymer, elastomer such as “nitrile” [copolymer of butadiene and acrylonitrile (NBR)] or “hydrogenated nitrile” (HNBR), plastic such a polyacetal (POM) or polybutylene terephtalate (PBT).

The solution S₁ may further comprise a solvent. This may be a protic solvent or aprotic solvent. It is preferable that the adhesion primer used should be soluble in the solvent of solution S₁.

By “protic solvent” under the present invention is meant a solvent which comprises at least one hydrogen atom able to be released in proton form.

The protic solvent is advantageously chosen from the group consisting of water, deionized water, distilled water, acidified or not, acetic acid, hydroxylated solvents such as methanol and ethanol, liquid glycols of low molecular weight such as ethyleneglycol, and their mixtures. In a first variant, the protic solvent used under the present invention only consists of one protic solvent or of a mixture of different protic solvents. In another variant, the protic solvent or mixture of protic solvents can be used in a mixture with at least one aprotic solvent, on the understanding that the resulting mixture has the characteristics of a protic solvent.

By “aprotic solvent” under the present invention is meant a solvent which is not considered to be protic. Said solvents are not able to release a proton or to accept a proton under non-extreme conditions.

The aprotic solvent is advantageously chosen from among dimethylformamide (DMF), acetone, tetrahydrofurane (THF), dichloromethane, acetonitrile, dimethyl sulfoxide (DMSO) and their mixtures.

If the solvent is a protic solvent, and advantageously when the adhesion primer is an aryl diazonium salt, the pH of the solution is typically less than 7. It is recommended to work at a pH of between 0 and 3 when the adhesion primer is prepared in the same medium as the grafting medium. If necessary the pH of the solution can be adjusted to the desired value using one or more acidifying agents well known to persons skilled in the art, for example using mineral or organic acids such as hydrochloric acid, sulphuric acid etc.

The adhesion primer can either be added as such to the solution S₁ as defined previously, or it can be prepared in situ in the solution. Therefore in one particular embodiment, the method of the present invention comprises a step to prepare the adhesion primer, notably when it is an aryl diazonium salt. Said compounds are generally prepared from arylamine, possibly containing several amine substituents by reaction with NaNO₂ in an acid medium or with NOBF₄ in an organic medium. For a detailed description of the experimental modes which can be used for said in situ preparation, the one skilled in the art may refer to the article by Belanger et al., 2006 (Chem. Mater., vol. 18, pages 4755-4763). Preferably, grafting is then conducted directly in the solution in which the aryl diazonium salt is prepared.

The solution S₁ may additionally contain at least one surfactant, in particular to improve the solubility of the elements it contains. A precise description of the surfactants that can be used under the invention is given in patent application FR 2 897 876 to which the one skilled in the art may refer. A single surfactant or a mixture of several surfactants may be used.

By “non-electrochemical conditions” used at step (b₁) of the method according to the invention, and in the meaning of the invention, is meant without any electric voltage. Therefore the non-electrochemical conditions used at step (b₁) of the method according to the invention are conditions which allow the formation of radical entities from the adhesion primer, in the absence of the application of any electric voltage to the surface on which the organic film is grafted. These conditions imply parameters such as temperature, type of solvent, presence of a particular additive, agitation, pressure, whereas electric current is not involved during the formation of the radical entities. Non-electrochemical conditions allowing the formation of radical entities are numerous, and this type of reaction is known and examined in detail in the prior art (Rempp & Merrill, Polymer Synthesis, 1991, 65-86, Hüthig & Wepf).

It is therefore possible for example to act on the thermal, kinetic, chemical, photochemical or radiochemical environment of the adhesion primer to cause its de-stabilization so that it forms a radical entity. It is evidently possible to act simultaneously on several of these parameters.

Under the present invention, the non-electrochemical conditions enabling the formation of radical entities are typically chosen from the group consisting of thermal, kinetic, chemical, photochemical, radiochemical conditions and their combinations. Advantageously, the non-electrochemical conditions are chosen from the group consisting of thermal, chemical, photochemical, radiochemical conditions and their combinations either together and/or with kinetic conditions. The non-electrochemical conditions used under the present invention are more particularly chemical conditions.

The thermal environment is a function of temperature. It can easily be controlled by applying heating means usually used by the one skilled in the art. The use of an environment with controlled temperature is of particular interest since it allows precise control over the reaction conditions.

The kinetic environment essentially corresponds to agitation of the system and to friction forces. It is not the agitation inside the molecules that is concerned here (elongation of bonds, etc.) but the global movement of the molecules. The application of pressure notably allows energy to be provided to the system so that the adhesion primer is de-stabilized and is able to form reactive species, notably radical species.

Finally, the action of different radiations such as electromagnetic radiation, γ radiation, UV radiation, electron beams or ion beams, may also de-stabilize the adhesion primer sufficiently so that it forms radicals and/or ions. The wavelength applied is chosen in relation to the primer used. For example, a wavelength of around 306 nm will be used for carried 4-hexylbenzenediazonium.

Regarding chemical conditions, one or more chemical initiators are used in the reaction medium. The presence of chemical initiators is often coupled with non-chemical environmental conditions such as set forth above. Typically, a chemical initiator will act on the adhesion primer and will generate the formation of radical entities from the latter. It is also possible to use chemical initiators whose action is not essentially related to environmental conditions and which can act over very broad ranges of thermal or even kinetic conditions. The initiator is preferably adapted to the environment of the reaction, for example to the solvent.

There are numerous chemical initiators. In general, a distinction is made between three types in relation to the environmental conditions used:
thermal initiators of which the most common are peroxides or azo compounds. Under the action of heat these compounds dissociate into free radicals. In this case, the reaction is conducted at a minimum temperature corresponding to the temperature required for the formation of radicals from the initiator. In general, these types of chemical initiators are used specifically within a certain temperature range, in relation to their decomposition kinetics;
photochemical or radiochemical initiators which are excited by the radiation triggered by irradiation (most often by UV, but also by γ radiation or by electron beams) which allow the production of radicals via more or less complex mechanisms. Bu₃SnH and I₂ belong to photochemical or radiochemical initiators;
essentially chemical initiators, these types of initiators acting rapidly and under normal temperature and pressure conditions on the adhesion primer to enable it to form radicals and/or ions. Said initiators generally have a redox potential which is lower than the reduction potential of the adhesion primer used under the reaction conditions. Depending on the type of initiator, it may for example be a reducing metal, such as iron, zinc, nickel; a metallocene such as ferrocene; an organic reducer such as hypophosphorous acid (H₃PO₂) or ascorbic acid; an organic or inorganic base in sufficient proportions to allow de-stabilization of the adhesion primer. Advantageously, the reducing metal used as chemical initiator is in finely divided form such as metal wool or metal filings. In general, when an organic or inorganic base is used as chemical initiator, a pH of 4 or more is generally sufficient. Structures of radical reservoir type such as polymeric matrices irradiated by a beam of electrons or a beam of heavy ions and/or by all the irradiation means previously cited can also be used as chemical initiators to destabilize the adhesion primer leading in particular to the formation of radical entities from this primer.

It is helpful to refer to the article by Mévellec et al., 2007 (Chem. Mater., vol. 19, pages 6323-6330) for the formation of active species.

The thickness of the organic film can easily be controlled, irrespective of the variant of the method of the present invention applied, as explained above. For each of the parameters such as duration of step (b₁) and depending on the reagents used, the one skilled in the art, via iteration, will be able to determine the optimal conditions to obtain a film of given thickness.

Advantageously, the method of the present invention may comprise an additional step, prior to grafting, to clean the surface on which it is desired to form the organic film, notably by sanding and/or polishing. Additional treatment using ultrasound with an organic solvent such as ethanol, acetone or dimethylformamide (DMF) is even recommended.

Similarly, the method of the present invention may comprise an additional step after grafting, consisting of subjecting the grafted organic film to heat treatment. Advantageously, said heat treatment consists of subjecting said grafted film to a temperature of between 60 and 180° C., notably between 90 and 150° C. and in particular in the order of 120° C. (i.e. 120° C.±10° C.) for a time of between 1 h and 3 days, notably between 6 h and 2 days and in particular between 12 and 24 h. This heat treatment step can be applied in a drying chamber or an oven.

Step (b) of the method according to the present invention consists of impregnating the polymer organic film grafted at step (a). Under the present invention, the term “impregnation” corresponds to the contacting of a film, such as previously defined, with a lubricating composition.

The lubricating composition comprises at least one lubricating molecule. The lubricating molecules typically correspond to natural or synthetic macromolecules, mineral, animal or vegetable oils. The lubricating molecules are preferably chosen from among synthetic molecules.

Amongst the macromolecules, mention may be made of polysiloxanes such as polydimethylsiloxane (PDMS), polydimethylsiloxane methacrylate (PDMSm) and octamethylcyclotetrasiloxane, or polyethers such as polyalkyleneglycols (PAG), and in particular polyethyleneglycol (POE), polypropyleneglycol (PPG) and polytetramethyleneglycol (PTMG). Advantageously, the lubricating molecule is chosen from among vinyl terminal hydrophilic macromolecules such as defined previously, or without vinyl terminal.

The composition may also comprise a film-swelling solvent, to facilitate penetration of the lubricating molecule into the film, and additives notably those use in the pharmaceutical, food or cosmetic industries.

The lubricating compositions of liquid type are preferred. Contacting may be achieved by dipping the surface into the composition or by spraying the surface with the composition. Impregnation is generally carried out until saturation of the film, i.e. it is no longer able to absorb the lubricating composition. If the composition comprises a swelling solvent, it is recommended to conduct the impregnating step several times to arrive at the saturation concentration in the film.

After step (b) of the method according to the invention, a grafted polymer gel is obtained. In the meaning of the invention a “grafted polymer gel” corresponds to the structure formed by the organic film grafted on the surface such as defined previously, impregnated with the lubricating composition such as defined previously.

The method may further comprise a rinsing step after impregnation to remove lubricating molecules directly present on the film surface. Preferably a solvent is used which does not cause the polymer film to swell.

The invention also concerns a lubrication kit to lubricate the surface of a solid and use thereof, said kit comprising:
in a first compartment, at least one adhesion primer and notably such as previously defined;
in a second compartment, at least one radically polymerizable hydrophilic monomer, notably such as previously defined;
optionally, in a third compartment, a chemical initiator for polymerization and notably such as previously defined;
optionally, in a fourth compartment, a lubricating composition such as previously defined.

In the kit of the present invention, the adhesion primer of the first compartment and the element of the second compartment may be in solution. Said solution corresponds more particularly to solution S₁ containing the initiator and the hydrophilic monomer, notably such as previously defined. The chemical initiator in the third compartment may also be in solution. Advantageously, an identical or different solvent is contained in each of the solutions of the first and second compartments, and optionally in the solution of the third compartment.

In one variant of the kit according to the invention, the first compartment does not contain an adhesion primer advantageously in solution, but at least one precursor of an adhesion primer advantageously in solution. In the meaning of the invention “adhesion primer precursor” is meant to designate a molecule separated from the primer by a single operative step that is easy to implement. In this case, the kit optionally comprises at least one other compartment in which there is at least one element necessary to prepare the primer from its precursor. Therefore the kit, for example, may contain an arylamine, i.e. the precursor of the adhesion primer, advantageously in solution, and also a solution of NaNO₂ or a solution of NOBF₄ to be added to allow the formation of an aryl diazonium salt i.e. the adhesion primer. The one skilled in the art will have understood that the use of a precursor means that the storage or transport of chemically reactive species can be avoided.

The solutions of the different compartments can evidently contain other agents, either the same or different, such as stabilizing agents or surfactants. The use of the kit proves to be simple, since all that is required is to place the sample whose surface is to be lubricated in contact with the mixture of solutions prepared extemporaneously by mixing the solutions of the different compartments, with the exception of the solution in the fourth compartment (lubricating composition), preferably under agitation and notably using ultrasound. Advantageously, only the solution containing the monomer i.e. in the second compartment is subjected to ultrasound before being mixed with the solution containing the adhesion primer either prepared extemporaneously from a precursor or present in the first compartment.

The invention also concerns the solid substrates treated with the lubricating method, and notably pairs of solid elements having interaction surfaces, at least one of said surfaces being lubricated with the method. Therefore, the present invention concerns a solid whose surface has been lubricated according to a method such as previously defined, and in which the step to impregnate the grafted organic film with a lubricating composition is not optional. The solid of the invention therefore has a grafted polymer gel on its surface, such as previously defined.

The invention is particularly adapted to solids and the mechanical systems used according to the invention, and more particularly their interaction surfaces, in a moist air environment notably with saturating water vapour, in an aqueous liquid or in biological media.

In particular the invention concerns mechanical systems treated or lubricated with the method of the invention or comprising a solid according to the present invention, and notably the different elements under friction which they contain, in particular systems of milli- or centimetre size; microelectromechanical systems (MEMS); spray mechanisms, catheter surfaces; precision mechanisms (clock movements); prostheses; polymer-based blood vessel substitutes; biochips whether or not integrated in vivo; powder dispensing systems; fluid dispensing systems and in particular biological fluids such as blood, plasma, lymph; inhalers . . . . The mechanical systems according to the invention and notably the different elements under friction which they contain may have on their surface either a grafted polymer or a grafted polymer gel such as previously defined.

### DETAILED DESCRIPTION OF SPECIFIC EMBODIMENTS

The following examples were conducted in a glass vessel. Unless otherwise specified, they were conducted under normal temperature and pressure conditions (around 25° C. under about 1 atm) in ambient air. Unless otherwise mentioned, the reagents used were commercial reagents used directly without any additional purification. The gold, plastic and glass slides used had a surface area of 1 cm².

### I—Grafting a Homogeneous Film by Chemical Grafting

### Example 1.1

### Grafting a pHEMA Film by Chemical Grafting

4-aminobenzoic acid (0.672 g, 4.9 10⁻³ mol) was solubilized in a solution of hydrochloric acid (50 mL at 0.5 M). To this solution were added 50 mL of NaNO₂ solution (0.338 g, 4.9 10⁻³ mol) in water. To this diazonium salt solution 1.6 mL of 2-hydroxyethylmethyl methacrylate (HEMA) (1.3 10⁻² mol) was added, followed by 6.5 mL of hypophosphorous acid solution (H₂PO₂) (63 10⁻³ mol).

After a reaction time of 3 min, the plastic samples and a reference gold slide for IR verification of the efficacy of the bath were placed in the reaction medium for 15 min. The different samples were successively rinsed in MilliQ water, ethanol then acetone for the gold slides before being dried.

IR spectrometry analysis of the gold slide confirmed that the grafting bath was active. The bands specific to pHEMA film at 3423 cm⁻¹ (OH deformation), 1729 cm⁻¹ (C═O deformation) and 1163 cm⁻¹ (C—O deformation) were present.

### Example 1.2

### Grafting a Poly(Acrylic Acid) (PAA) Film by Chemical Grafting

The operating mode is identical to the mode in Example 1.1. The 2-hydroxyethylmethyl methacrylate was substituted by acrylic acid (AA) (0.91 mL, 1.3 10⁻² mol).

IR spectrometry analysis of the gold slide confirmed that the grafting bath was active. The bands specific to PAA film at 3356 cm⁻¹ (COOH deformation), 1710 cm⁻¹ (C═O deformation) and 1265 cm⁻¹ (C—O deformation) were present.

### Example 1.3

### Grafting a Polypropylene Glycol (PPG) Film by Chemical Grafting

4-aminobenzoic acid (0.336 g, 2.4 10⁻³ mol) was solubilized in a solution of hydrochloric acid (25 mL at 0.5 M). To this solution were added 25 mL of NaNO₂ solution (0.169 g, 2.4 10⁻³ mol) in water. To this diazonium salt solution were added 2.5 mL of polypropylene glycol methacrylate (PPGm) (6.7 10⁻³ mol) then 3.2 mL of hypophosphorous acid solution (H₃PO₂) (32 10⁻³ mol).

After a reaction time of 3 min, the plastic samples and a reference gold slide for IR verification of bath efficacy were placed in the reaction medium for 15 min. The different samples were successively rinsed with MilliQ water, ethanol and then acetone for the gold slide before being dried.

IR spectrometry analysis of the gold slide confirmed that the grafting bath was active. The bands specific to PPG film at 3451 cm⁻¹ (OH deformation), 1720 cm⁻¹ (C═O deformation) and 1112 cm⁻¹ (C—O deformation) were present.

### II—Grafting a Heterogeneous Film by Chemical Grafting

### Example 2.1

### Grafting a pHEMA/PPG Copolymer Film by Chemical Grafting

The operating mode is identical to the mode in Example 1.4. The polypropylene glycol methacrylate (PPGm) was substituted by a mixture of 2-hydroxyethylmethyl methacrylate (HEMA) (0.8 mL, 6.7 10⁻³ mol) and polypropylene glycol methacrylate (PPGm) (2.5 mL, 6.7 10⁻³ mol).

IR spectrometry analysis of the gold slide confirmed that the grafting bath was active. The bands specific to pHEMA/PPG film at 3438 cm⁻¹ (OH deformation), 1729 cm⁻¹ (C═O deformation) and 1156 cm⁻¹ (C—O deformation) were present.

### Example 2.2

### Grafting a PPG/PEG copolymer Film by Chemical Grafting

The operating mode is identical to the mode in Example 1.1. The 2-hydroxyethylmethyl methacrylate was substituted by a mixture of PPGm (5.0 mL, 1.3 10⁻² mol) and polyethylene glycol methacrylate (PEGm) (4.4 mL, 1.4 10⁻² mol).

IR spectrometry analysis of the gold slide confirmed that the grafting bath was active. The bands specific to PPG/PEG film at 3443 cm⁻¹ (OH deformation), 1725 cm⁻¹ (C═O deformation) and 1097 cm⁻¹ (C—O deformation) were present.

### Example 2.3

### Grafting a PAA/PPG Copolymer Film by Chemical Grafting

The operating mode is identical to the mode in Example 1.1. 2-hydroxyethylmethyl methacrylate was substituted by a mixture of AA (0.91 mL, 1.3 10⁻² mol) and PPGm (5.0 mL, 1.3 10⁻² mol).

IR spectrometry analysis of the gold slide confirmed that the grafting bath was active. The bands specific to PAA/PPG film at 3442 cm⁻¹ (OH deformation), 1728 cm⁻¹ (C=0 deformation) and 1109 cm⁻¹ (C—O deformation) were present.

### III—Grafting a Homogeneous Film by Chemical Grafting+Impregnation

### Example 3.1

### Chemically Grafted Film of pHEMA+Silicon Oil Impregnation

The example was conducted following the conditions described in Example 1.1 to create the pHEMA film. The samples thus prepared were then placed in a bath of silicon oil for 15 min. The different samples were successively rinsed with MilliQ water, then with ethanol before being dried.

### Example 3.2

### Chemically Grafted pHEMA Film+PDMSm Impregnation

The example was conducted following the conditions described in Example 1.1 to create the pHEMA film. The samples thus prepared were then placed in a PDMSm bath for 15 min. The different samples were successively rinsed with MilliQ water then ethanol before being dried.

### Example 3.3

### Chemically Grafted pHEMA Film+PPGm Impregnation

The example was conducted following the conditions described in Example 1.1 to create the pHEMA film. The samples thus prepared were then placed in a PPGm bath for 15 min. The different samples were successively rinsed with MilliQ water then ethanol before being dried.

### Example 3.4

### Chemically Grafted PAA Film+PPGm Impregnation

The example was carried out following the conditions described in Example 1.2 to create the PAA film. The samples thus prepared were then placed in a PPGm bath for 15 min. The different samples were successively rinsed with MilliQ water then ethanol before being dried.

### IV—Films Having Lubricating Properties

Lubrication measurements were carried out using syringes and pistons. The syringes and pistons were slightly moistened before assembly. The piston was plunged once inside the syringe before starting to take measurements. The syringes were placed on a fixed block. The piston was subjected to a thrust force over 1 cm at a constant rate of 3.8 mm/min. The thrust force was measured using a strain gauge. The force values read on this apparatus were video-recorded. The diagrams of the applied thrust force as a function of time are described below.

After analysis, for those surfaces treated according to the method, no pollution of the environment was observed by the impregnated lubricating composition in the aqueous medium, and no degradation products. With the silicon oil, slight pollution of the medium by the oil could be observed.

By comparison, it was ascertained that under similar conditions, notably in an aqueous environment, the treatment of the samples with the method leads to better results in terms of lubrication than with silicon oil treatment (up to ten times for a pHEMA film impregnated with silicon). The thrust force required for equivalent displacement is greater for cases with silicon oil treatment than with treatment according to the method of the invention.

The results obtained during tests show that lubrication with the method substantially improves the tribological properties of treated surfaces without any substantial dispersion of lubricating molecules or degradation products.

1. Method for lubricating a surface of a solid, characterized in that it comprises the steps consisting of:
a) grafting on said surface a polymer organic film consisting of polymers grafted on said surface, each polymer having a first unit directly bonded to said surface, derived from a cleavable aryl salt, and at least one other unit of the polymer chain derived from a radically polymerizable hydrophilic monomer, and optionally,
b) impregnating the polymer organic film of step (a) with a lubricating composition.

2. Method for lubricating a surface of a solid, characterized in that it comprises the steps consisting of:
a) grafting on said surface a polymer organic film consisting of polymers grafted on said surface, each polymer having a first unit directly bonded to said surface, derived from a cleavable aryl salt, and at least one other unit of the polymer chain derived from a radically polymerizable hydrophilic monomer, and
b) impregnating the polymer organic film of step (a) with a lubricating composition.

3. Lubrication method according to claim 1 or 2, characterized in that said cleavable aryl salt is chosen from the group consisting of aryl diazonium salts, aryl ammonium salts, aryl phosphonium salts, aryl iodonium salts and arylsulfonium salts.

4. Lubrication method according to claim 1 or 2, characterized in that said cleavable aryl salt is a compound of formula (I):
R—N₂⁺,A⁻ (I)
wherein:
A represents a monovalent anion, and
R represents an aromatic or heteroaromatic carbon structure, optionally mono- or polysubstituted, consisting of one or more aromatic or heteroaromatic cycles each comprising 3 to 8 atoms, the heteroatom(s) possibly being N, O, P or S, the substituent(s) optionally containing one or more heteroatoms, C₁ to C₆ alkyl groups or C₄ to C₁₂ thioalkyl groups.

5. Lubrication method according to claim 1 or 2, characterized in that said hydrophilic monomer meets following formula (II): 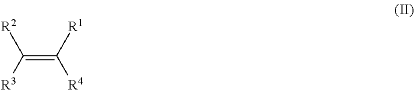
wherein the groups R₂ to R₄, the same or different, represent a non-metallic monovalent atom such as a halogen atom, a hydrogen atom, a saturated or unsaturated chemical group such as an alkyl, aryl group, a hydrophilic polymer chain, a nitrile, a carbonyl, an amine, an amide, a —COOR₅ group in which R₅ is a hydrogen atom or C₁-C₁₂ and preferably C₁-C₆ alkyl group, and in which the R₁ group represents a carboxylic acid, a hydrophilic polymer chain or —COOR₅ group in which R₅ is a hydrophilic polymer chain.

6. Lubrication method according to claim 1 or 2, characterized in that said hydrophilic monomer is chosen from among acrylic acid, hydroxyethyl methacrylate (HEMA) and polyalkyleneglycols (PAG) with vinyl terminal.

7. Lubrication method according to claim 1 or 2, characterized in that said film contains units derived from a first monomer of vinyl terminal hydrophilic macromolecule type, and units derived from a second monomer which is not a macromolecule with vinyl terminal.

8. Lubrication method according to any of claim 1 or 2, characterized in that said grafting is chemical grafting.

9. Lubrication method according to claim 1 or 2, characterized in that said method comprises the steps consisting of:
a₁) contacting said surface with a solution S₁ comprising at least one cleavable aryl salt and at least one radically polymerizable hydrophilic monomer;
b₁) subjecting said solution S₁ to non-electrochemical conditions allowing the formation of radical entities from said cleavable aryl salt.

10. Lubrication method according to claim 1 or 2, characterized in that there is an additional step, after grafting step (a), consisting of subjecting the grafted organic film to a temperature of between 60 and 180° C., notably between 90 and 150° C. and in particular in the order of 120° C. for a time of between 1 h and 3 days, notably between 6 h and 2 days and in particular between 12 and 24 h.

11. Lubrication method according to claim 1 or 2, characterized in that said lubricating composition comprises at least one lubricating molecule chosen from among natural or synthetic macromolecules, mineral, animal or vegetable oils.

12. Lubrication method according to claim 1 or 2, characterized in that said lubricating composition comprises at least one lubricating molecule chosen from among polysiloxanes or polyethers.

13. Lubrication kit to lubricate a surface of a solid, characterized in that it comprises:
in a first compartment, at least one cleavable aryl salt,
in a second compartment, at least one radically polymerizable hydrophilic monomer;
optionally, in a third compartment, a chemical initiator for polymerization;
optionally in a fourth compartment, a lubricating composition.

14. A Solid whose surface has been lubricated according to a method such as defined in claim 2.

15. A Mechanical system lubricated according to the method in claim 1 or 2, characterized in that said system is chosen from among systems of milli- or centimetric size, microelectromechanical systems (MEMS), spray mechanisms, catheter surfaces, precision mechanisms (clock movements), prostheses, polymer-based blood vessel substitutes, biochips whether or not integrated in vivo, powder dispensing systems, fluid dispensing systems and in particular of biological fluids, and inhalers.

16. Use of a substrate according to claim 14 in a moist air environment, in an aqueous liquid or in biological media.

17. A Mechanical system comprising a solid according to claim 14, characterized in that said system is chosen from among systems of milli- or centimetric size, microelectromechanical systems (MEMS), spray mechanisms, catheter surfaces, precision mechanisms (clock movements), prostheses, polymer-based blood vessel substitutes, biochips whether or not integrated in vivo, powder dispensing systems, fluid dispensing systems and in particular of biological fluids, and inhalers.

18. Use of a mechanical system according to claim 15 in a moist air environment, in an aqueous liquid or in biological media.
